(19) United States
(12) Patent Application Publication
(10) Pub. No.: US 2021/0315743 A1
(43) Pub. Date: Oct. 14, 2021

(54) DISPOSABLE DIAPER WITH REPLACEABLE INSERT

(71) Applicant: Albert Molhemanhar, Los Gatos, CA (US)

(72) Inventor: Albert Molhemanhar, Los Gatos, CA (US)

(21) Appl. No.: 16/846,281

(22) Filed: Apr. 11, 2020

(51) Int. Cl. A61F 13/49; A61F 13/58; A61F 13/505

(52) U.S. Cl. CPC A61F 13/4906; A61F 13/505; A61F 13/58

(57) ABSTRACT

A disposable diaper that includes a traditional outer shell and a replaceable insert is disclosed. The insert can be removed without taking the outer shell off the wearer. The insert may be replaced as many times as desired by the user, thereby greatly reducing the amount of waste associated with disposable diapers. It should also be noted that the disposable diaper may be manufactured and marketed as a unit, the unit including both the outer shell and the replaceable insert. Alternatively, the replaceable inserts may be manufactured and marketed as a separate product.

### CROSS REFERENCE TO RELATED APPLICATIONS

No related applications.

### FIELD OF THE DISCLOSURE

The present disclosure relates generally to disposable diapers, and more particularly is a disposable diaper with a replaceable insert.

### SUMMARY

In various embodiments of the present disclosure, a disposable diaper with a replaceable insert is presented. The disposable diaper includes an outer shell with a replaceable insert. The replaceable insert is made of an absorbent material. In various embodiments, the replaceable insert is removably secured to the outer shell of the diaper. The body of the replaceable insert may include a first component and a second component that overlap one another to prevent leakage. The replaceable insert is constructed with extending tabs that extend beyond the outer shell so that a user can grasp the tabs to pull the replaceable insert out of the outer shell when the replaceable insert is wet.

### BRIEF DESCRIPTION OF THE DRAWINGS

The accompanying drawings, wherein like reference numerals refer to identical or functionally similar elements throughout the separate views, together with the detailed description below, illustrate embodiments of concepts that include the claimed disclosure, and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 is a top plan view of the disposable diaper with replaceable insert in an open position and laid flat.

FIG. 2 is a top plan view showing the two components of the replaceable insert.

FIG. 3 shows the disposable diaper in approximately the position it would be in when worn by a user.

FIG. 4 shows an attaching element of the components of the replaceable insert.

FIG. 5 illustrates the surface of the replaceable insert that would be next to the skin of a user.

### DETAILED DESCRIPTION

The present disclosure is generally directed to a disposable diaper. The diaper includes a traditional outer shell. A replaceable insert is provided that allows a user to remove the insert without taking the shell off the wearer. The insert may be replaced as many times as desired by the user, until the outer shell is damaged.

Having a disposable diaper with an outer shell that may be used multiple times greatly reduces the amount of waste associated with disposable diapers. At each changing of a wet diaper, the user need dispose only of the replaceable insert, which is made from far less material than is the outer shell. This significantly reduces the amount of waste bound for landfills, thereby aiding preservation of the environment.

Moreover, the replaceable insert is less expensive to manufacture than is a traditional disposable diaper. Therefore the user will recognize significant cost savings in addition to the environmental benefits. It should be noted that the disposable diaper may be manufactured and marketed as a unit, the unit including both the outer shell and the replaceable insert. Alternatively, the replaceable inserts may be manufactured and marketed as a separate product.

It should also be noted that the construction of the disposable diaper disclosed herein makes it very easy to change diapers. All that need be done is for the user to pull the tabs, pulling the replaceable insert out of the outer shell. The wet replaceable insert is thus removed, thereby temporarily eliminating the need to even remove the outer shell from the wearer.

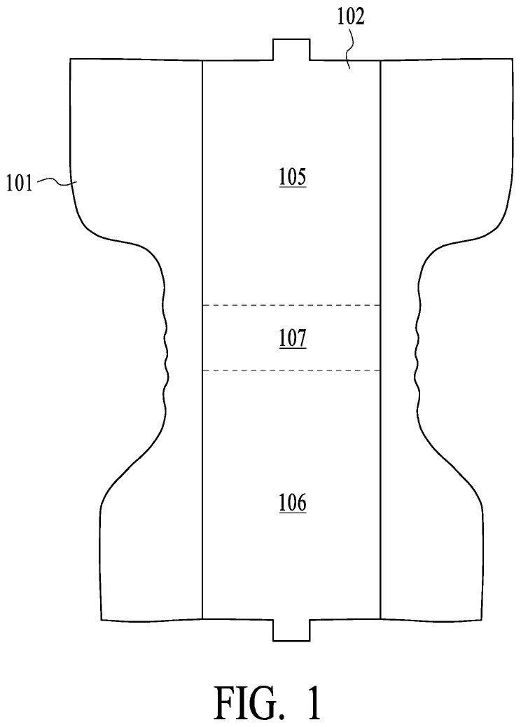

Referring first to FIG. 1, the present disclosure is directed to a disposable diaper 100. The disposable diaper 100 includes an outer shell 101. The outer shell 101 is formed to conform to the body of the intended wearer. Therefore, it is envisioned that the outer shell 101 will be manufactured in a variety of sizes. The outer shell 101 can be manufactured with known processes for producing disposable diapers. The outer shell 101 will typically be made with a waterproof outer layer and an absorbent inner layer. Those skilled in the art will recognize that various materials will suffice to provide these intended characteristics.

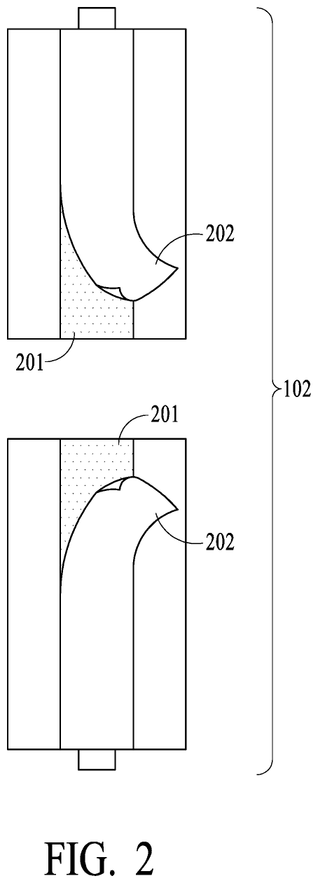

The diaper 100 further includes a replaceable insert 102. The replaceable insert 102 is made of absorbent material so as to absorb liquid waste to keep it from irritating the skin of the wearer. The replaceable insert 102 may include a mechanism to secure the replaceable insert 102 in place in the outer shell 101. While nearly any securing mechanism will do, in many embodiments, an adhesive attaching element 201 is utilized as illustrated in FIG. 2. The attaching element 201 may extend for any fraction of the length of the replaceable insert 102. The attaching element 201 may be only a relatively small element so as not to render the replaceable insert 102 unduly difficult to remove from the outer shell 101. Besides adhesive, other methods of attaching the replaceable insert 102 to the outer shell 101 will be readily apparent to those of ordinary skill in the art.

The attaching element 201 is secured to the outer surface of the replaceable insert 102. A non-adhesive cover 202 may cover the attaching element 201 to avoid the attaching element 201 adhering to surfaces it is not intended to be affixed to. When the user is ready to deploy the insert 102, the cover 202 is removed and the replaceable insert 102 is positioned as desired in the outer shell 101, with the attaching element 201 adhering to the inner surface of the outer shell 101.

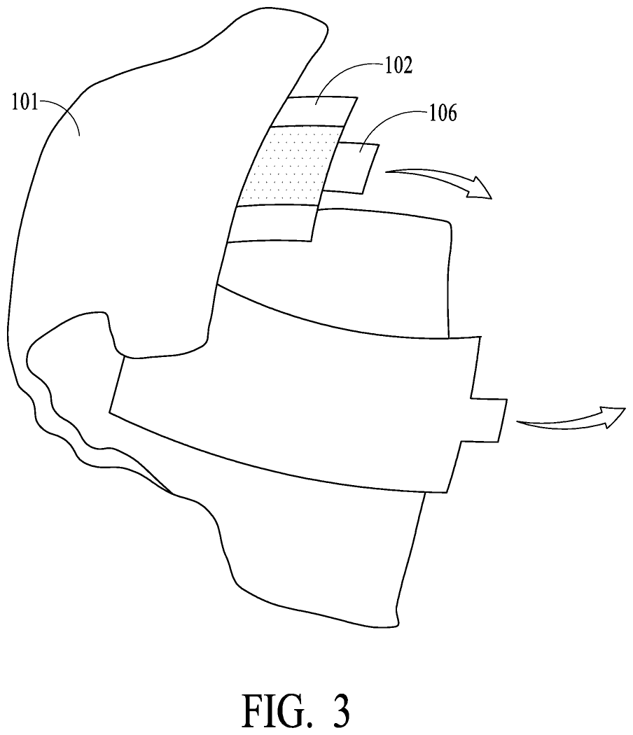

In various embodiments, in order to facilitate the easy removal of the replaceable insert 102 from the outer shell 101, the replaceable insert 102 includes two pieces, a first component 103 and a second component 104. In some embodiments, the first and second components 103, 104 are positioned so as to form an overlapping region 105. The overlapping region 105 ensures that moisture does not escape the replaceable insert 102 when the diaper 100 is in use. Moisture is thereby prevented from spreading to or escaping from the outer shell 101. An embodiment with the replaceable insert 102 positioned in the outer shell 101 is shown in FIG. 3.

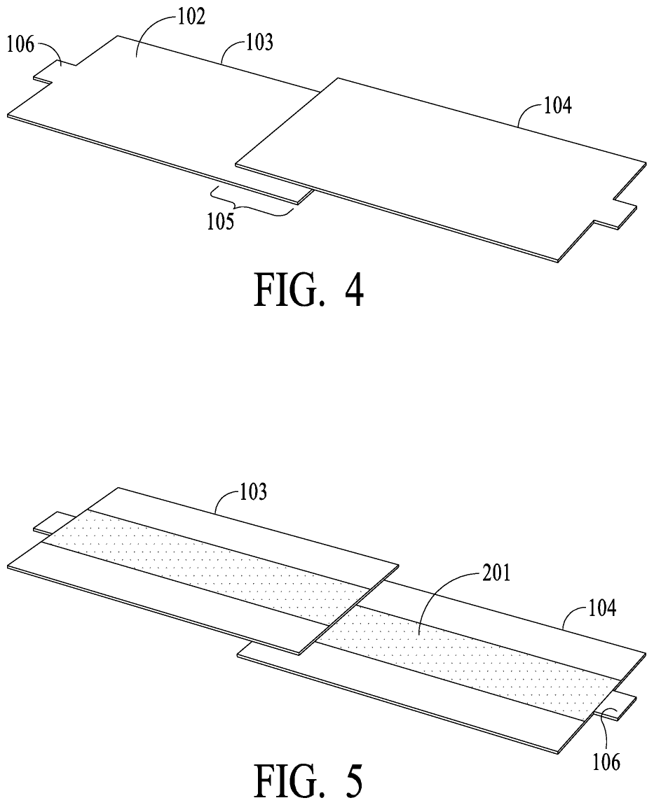

The replaceable insert 102 is provided with tabs 106 at each distal end. The tabs 106 are positioned so that they extend beyond the profile of the outer shell 101. The user grasps the tabs 106 and pulls to remove the replaceable insert 102 without removing the diaper 100 from the wearer. This provides a quick and easy method of removing the wet material of the insert 102 from the diaper 100 without requiring the removal of the outer shell 101 from the wearer. The inner surface of the replaceable insert 102, the surface next to the wearer, is illustrated in FIG. 4. The outer surface of the insert 102, the surface in contact with the outer shell 101, is shown in FIG. 5. Note that the attaching element 201 is required only on the outer surface of the insert 102.

The terminology used herein is for the purpose of describing particular embodiments only and is not intended to be limiting of the technology. As used herein, the singular forms “a”, “an” and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present disclosure. As such, some of the components may have been distorted from their actual scale for pictorial clarity.

In the foregoing description, for purposes of explanation and not limitation, specific details are set forth, such as particular embodiments, procedures, techniques, etc. in order to provide a thorough understanding of the present invention. However, it will be apparent to one skilled in the art that the present invention may be practiced in other embodiments that depart from these specific details.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. A disposable diaper, comprising:
an outer shell; and
a replaceable insert, the replaceable insert comprising absorbent material, the replaceable insert being removably secured to the outer shell of the diaper.

2. The diaper of claim 1, wherein:
the body of the replaceable insert comprises a first component and a second component.

3. The diaper of claim 2, wherein:
the first and second components of the replaceable insert are positioned so as to form an overlapping region when the replaceable insert is installed in the outer shell.

4. The diaper of claim 1, wherein:
the replaceable insert comprises tabs that extend beyond the outer shell so that a user can grasp the tabs to pull the replaceable insert out of the outer shell when the replaceable insert is wet.

5. The diaper of claim 1, wherein:
the replaceable insert is secured to the outer shell.

6. The diaper of claim 1, wherein:
the replaceable insert is secured to the outer shell via adhesive.

7. The diaper of claim 6, wherein:
the adhesive is covered by a removable non-adhesive cover to facilitate storage and transport of the replaceable insert before insertion of the replaceable insert into the outer shell.

8. A disposable diaper, comprising:
an outer shell; and
a replaceable insert, the replaceable insert comprising absorbent material, the replaceable insert being removably secured to the outer shell of the diaper, and the replaceable insert comprising two overlapping components.

9. The diaper of claim 8, wherein:
the replaceable insert comprises tabs that extend beyond the outer shell so that a user can grasp the tabs to pull the replaceable insert out of the outer shell when the replaceable insert is wet.

10. The diaper of claim 8, wherein:
the replaceable insert is secured to the outer shell by adhesive.

11. The diaper of claim 10, wherein:
a removable non-adhesive cover covers the adhesive for storage and transport prior to the installation of the replaceable insert into the outer shell.

12. A disposable diaper, comprising:
an outer shell;
a replaceable insert, the replaceable insert comprising absorbent material, the replaceable insert being removably secured to the outer shell of the diaper; and
tabs on the replaceable insert that extend beyond the outer shell so that a user can grasp the tabs to pull the replaceable insert out of the outer shell when the replaceable insert is wet.

13. The diaper of claim 12, wherein:
the body of the replaceable insert comprises a first component and a second component, the first and second components positioned so as to form an overlapping region.

14. The diaper of claim 12, wherein:
the replaceable insert is secured to the outer shell.

15. The diaper of claim 14, wherein:
the replaceable insert is secured to the outer shell by adhesive.

16. The diaper of claim 15, wherein:
The adhesive is covered by a removable non-adhesive cover for storage and transport of the replaceable insert prior to the replaceable insert being inserted into the outer shell.
